(19) United States
(12) Patent Application Publication
(10) Pub. No.: US 2010/0071702 A1
(43) Pub. Date: Mar. 25, 2010

(54) SEXUALLY TRANSMITTED DISEASE SHIELD AND CONDOM

(76) Inventor: Douglas J. Sturlingh, Clearwater, FL (US)

Correspondence Address: LARSON AND LARSON, 11199 69TH STREET NORTH, LARGO, FL, 33773, US

(21) Appl. No.: 12/235,278

(22) Filed: Sep. 22, 2008

(51) Int. Cl. A61F 6/02

(52) U.S. Cl. 128/844; 128/842

(57) ABSTRACT

An application for a system for reducing the risk of pregnancy and sexually transmitted diseases includes a condom with a thicker wall towards the base for tear-resistance and a thinner wall towards the tip where sensitivity is important. The condom also includes a resilient ring at the base for improved retention and performance. The system for reducing the risk of pregnancy and sexually transmitted diseases also includes a shield portion that rests against the wearer's pubic area, covering the pubic area and scrotum to protect them from fluids that, in some circumstances, contain sexually transmitted disease virus. The shield has a retention ring. The resilient ring inserts into and is removably held within the retention ring of the shield.

### CROSS-REFERENCE TO RELATED APPLICATION

This application is related to U.S. application titled, “CONDOM,” which was filed on even date herewith; attorney docket number 2589.0 and inventor Douglas J. Sturlingh.

### FIELD OF THE INVENTION

This invention relates to a birth control and disease preventing device, and more particularly to a device that reduces the risk of both pregnancy and the transmission of sexually transmitted diseases.

### BACKGROUND OF THE INVENTION

Various methods exist for birth control such as the birth control pill, the condom and the inter-uterine device (IUD). The condom is a simple, low-cost method with rare side effects. When properly used, a condom can aid in the prevention of pregnancy as well as preventing the transmission of sexual diseases.

Condoms come in various sizes, shapes, textures and materials. Some condoms come rolled and some not rolled.

Unfortunately, condoms are not 100% effective against pregnancy and the transmission of sexually transmitted disease. Pregnancy often occurs due to a tear in a condom or if a condom falls off. The same often leads to the transmission of sexually transmitted diseases. Additionally, sexually transmitted diseases can be transmitted in fluids passing from one partner to the other onto peripheral areas such as the scrotum or base of the penis.

What is needed is a condom and shield with improved performance in birth control and disease prevention.

### SUMMARY OF THE INVENTION

A system for reducing the risk of pregnancy and sexually transmitted diseases includes a condom portion with a thicker wall towards the base for tear-resistance and a thinner wall towards the tip where sensitivity is important. The condom also includes a resilient ring at the base for improved retention and performance. The system for reducing the risk of pregnancy and sexually transmitted diseases also includes a shield portion that rests against the wearer's pubic area, covering the pubic area and scrotum to protect them from fluids that, in some circumstances, contain sexually transmitted disease virus. The shield has a retention ring. The resilient ring inserts into and is removably held within the retention ring of the shield.

In one embodiment, a system for reducing the risk of pregnancy and sexually transmitted diseases is disclosed including a body that has walls made of a material. The walls have a base end, a tip end and a circumference at the base end. The material is thicker at the base end of the wall and thinner at an area of the tip end of the wall and the wall open at the base end for insertion of a penis and closed at a distal end. A pressure ring is formed around the circumference of the base end. The system for reducing the risk of pregnancy and sexually transmitted diseases includes a shield that has a circular opening for accepting the penis and a retention ring formed around the circular opening such that the pressure ring of the condom presses into the retention ring of the shield and the pressure ring of the condom is removably held within the retention ring of the shield for the prevention of pregnancy and sexually transmitted disease during intercourse.

In another embodiment, a method of reducing the risk of pregnancy and sexually transmitted diseases is disclosed. The method includes providing a condom that has a body that has walls made of a material. The walls have a base end, a tip end and a circumference at the base end. The material is thicker at the base end of the wall and thinner at an area of the tip end of the wall and the wall open at the base end for insertion of a penis and closed at a distal end. A pressure ring is formed around the circumference of the base end. The method continues with, before engaging in sex, pulling the shield over the shaft of one's penis so that the retention ring faces away from the wearer then pulling the condom over the penis such that the pressure ring and base end covers a base portion of the penis and the tip end covers the tip portion of the penis then engaging the pressure ring into the retention ring.

In another embodiment, a system for preventing pregnancy and sexually transmitted diseases includes a condom having a body. The body has a wall made of a material. The wall has a base end, a tip end and a circumference at the base end. The material has a first thickness at the base end of the wall and a second thickness at an area of the tip end of the wall and the first thickness is three times the second thickness. The wall is open at the base end for insertion of a penis and closed at a distal end. A pressure ring is formed at the base end of the wall around the circumference of the base end. The system for preventing pregnancy and sexually transmitted diseases also includes a shield that has a circular opening for accepting the penis and a retention ring formed around the circular opening. The pressure ring of the condom engages with the retention ring of the shield and the pressure ring of the condom is removably held within the retention ring of the shield for the prevention of pregnancy and sexually transmitted disease during intercourse.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a perspective view of a shield of the present invention.

FIG. 2A illustrates a side view of a first interface between the shield and the condom of the present invention.

FIG. 2B illustrates a side view of the first interface between the shield and the condom of the present invention.

FIG. 3A illustrates a side view of a second interface between the shield and the condom of the present invention.

FIG. 3B illustrates a side view of the second interface between the shield and the condom of the present invention.

FIG. 4 illustrates a side cut-away view of the present invention worn by male person.

FIG. 5 illustrates a front perspective view of the present invention worn by male person.

### DETAILED DESCRIPTION OF THE INVENTION

Reference will now be made in detail to the presently preferred embodiments of the invention, examples of which are illustrated in the accompanying drawings. Throughout the following detailed description, the same reference numerals refer to the same elements in all figures.

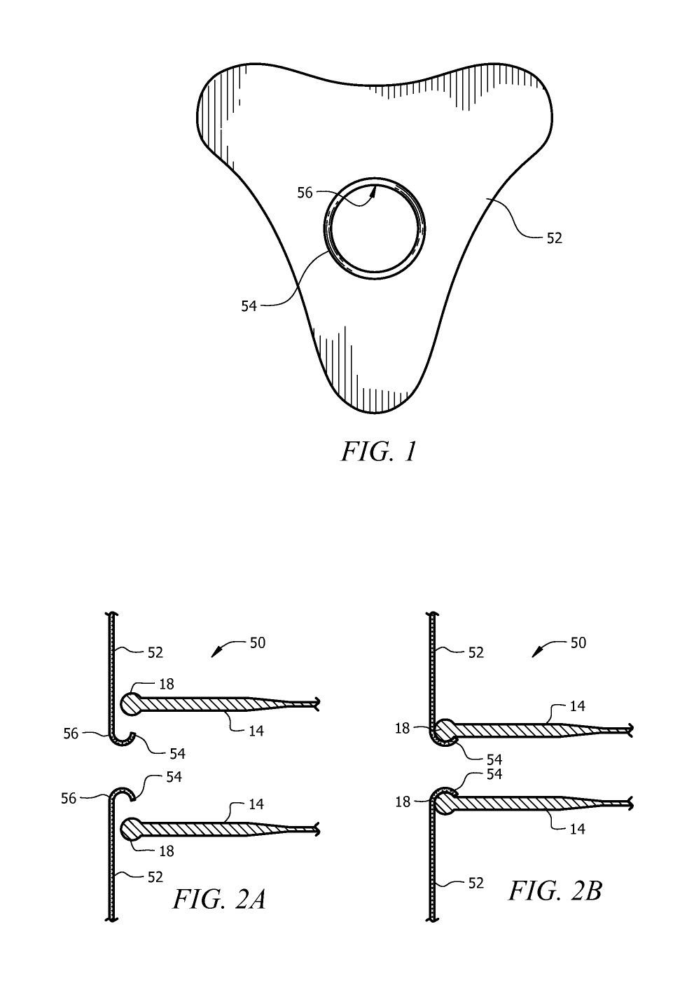

Referring to FIG. 1, a perspective view of a shield of the present invention is shown. The shield 52 is made from a stiff, yet pliable material such as latex. It is stiff enough to maintain its shape and pliable enough to bend upon pressure from the wearer. The shield has an opening bordered by an inner edge of a retention ring 56 through which the penis of the wearer passes. The retention ring 54/56 forms a resilient clip for accepting and holding a pressure ring 18 of a condom 10 (see FIGS. 2-4). The shield 52 is formed to cover the male pubic area including the scrotum.

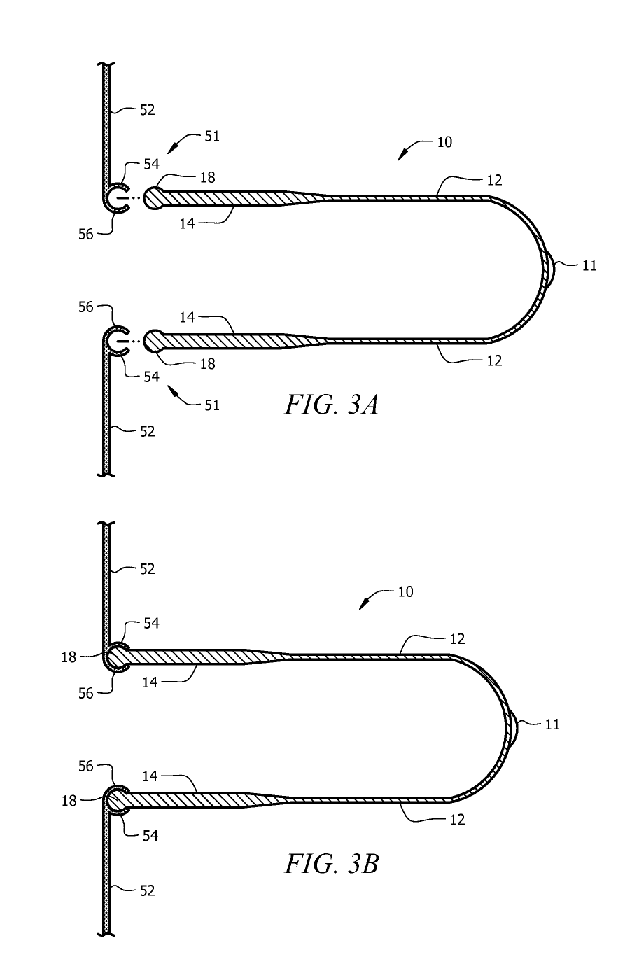

Referring to FIGS. 2A and 2B, a side view of a first interface between the shield 52 and the condom 10 of the present invention is shown. In this example, the first interface 50 has a side oriented retention ring 54/56. In this, the retention ring 54/56 is formed as part of the shield 52. The retention ring 54/56 accepts and holds the pressure ring 18 of the condom 10 (see FIGS. 3A and 3B). The pressure ring 18 is formed on the thicker wall surface 14 of the condom 10. In FIG. 2A, the pressure ring 18 is shown stretched and ready to mate with the retention ring 54/56 while in FIG. 2B, the pressure ring 18 is held securely in the retention ring 54/56. In some embodiments, the inner circumference of the pressure ring 18 is less than the outer circumference of the retention ring 54 and the resiliency of the pressure ring 18 holds the pressure ring 18 against the retention ring 54/56. In some embodiments, the opening of the retention ring 54/56 is smaller than the cross-sectional diameter of the pressure ring 18, thereby opening to accept the pressure ring 18 and closing to hold the pressure ring 18 securely.

Referring to FIGS. 3A and 3B, a side view of a second interface between the shield 52 and the condom 10 of the present invention is shown. In this example of the second interface 51, the retention ring 54/56 faces outward towards the condom 10 and is ready to accept and hold the pressure ring 18. The opening of the retention ring 54/56 is smaller than the cross-sectional diameter of the pressure ring 18. The retention ring 54/56 opens slightly to accept and hold the pressure ring 18 by way of a curved surface that engages around the outer periphery of the pressure ring 18 similar to the seal of a common plastic bag (zipper lock, etc.) as shown in FIG. 3B.

The tip area of the condom 10 has a thinner, closed wall 12 to cover the tip of the penis for greater sensitivity and a thicker wall 14 towards the base of the condom for added strength. At the very base of the condom 10 is the pressure ring 18. In some embodiments, the thicker wall 14 of the condom 10 is three times thicker than the thinner wall 12. Formed at the base of the condom 10 is the pressure ring 18. The pressure ring 18 serves as a seal to lock into the shield 52. In addition, the pressure ring 18 serves to help prevent the condom 10 from falling off during sex and serves to constrict blood flow within the penis providing a longer lasting erection. In some embodiments, a receptacle 11 is formed in the tip of the condom 10 to provide a space for semen to flow. It is anticipated that the condom 10 is made from materials known in the industry such as latex, rubber and natural materials from animals such as lambskin.

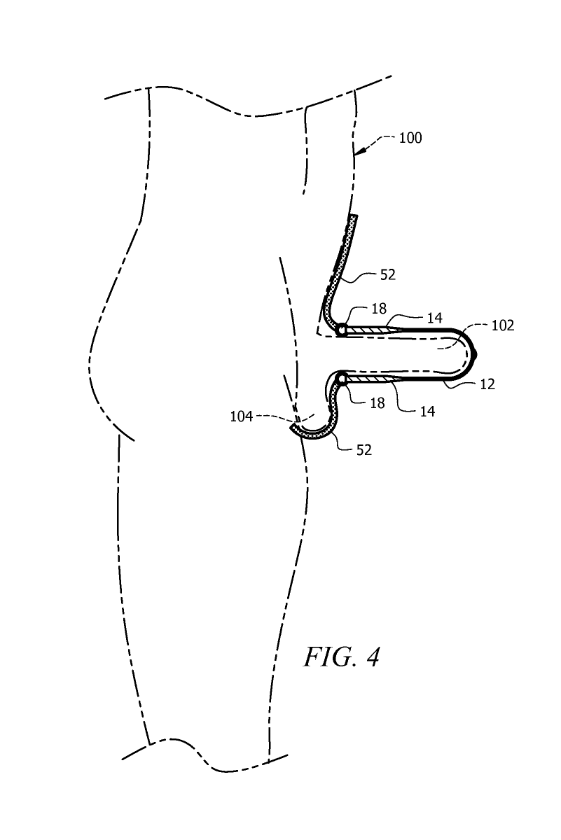

Referring to FIG. 4, a side cut-away view of the present invention worn by male person is shown. Although the condom 10 and the shield 52 are independent and can be used without the other, the best overall protection is provided when used as shown in FIG. 4. First, the shield 52 is passed over the penis 102 and rests against the wearer's body 100, covering the scrotum 104. Next, the condom 10 is pulled over the penis 102 and the pressure ring 18 is engaged with the retention ring 54/56, sealing the wearer's 100 genitals from the intended partner. As shown, the base 14 of the condom 10 is thicker than the tip area 12 of the condom 10. This provides greater tear resistance at the base of the condom 10 while providing greater sensitivity at the tip area 12.

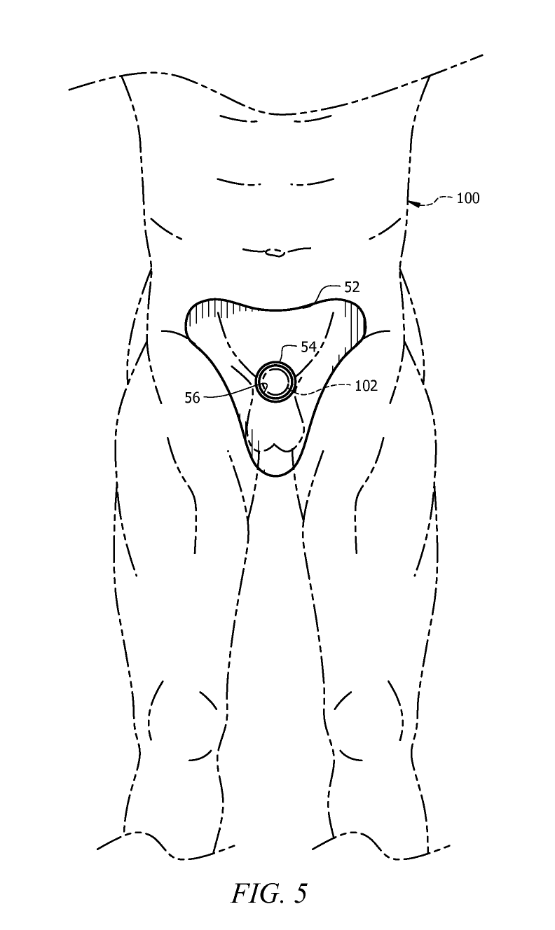

Referring to FIG. 5, a front perspective view of the present invention worn by male person is shown. The shield 52 covers much of the genital area and scrotum. The retention ring 54/56 is shown ready to accept the pressure ring 18 of the condom 10.

Equivalent elements can be substituted for the ones set forth above such that they perform in substantially the same manner in substantially the same way for achieving substantially the same result.

It is believed that the system and method of the present invention and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A sexually transmitted disease shielding system comprising:
a condom having a body, the body having a wall made of a material, the wall having a base end, a tip end and a circumference at the base end, the material is thicker at the base end of the wall and the material is thinner at an area of the tip end of the wall, the wall is open at the base end for insertion of a penis and the wall is closed at a distal end; a pressure ring is formed around the circumference of the base end; and
a shield, the shield having a circular opening for accepting the penis and the shield having a retention ring formed around the circular opening;
whereas, the pressure ring of the condom presses into the retention ring of the shield and the pressure ring of the condom is removably held within the retention ring of the shield for the prevention of pregnancy and sexually transmitted disease during intercourse.

2. The sexually transmitted disease shielding system of claim 1, wherein material is rubber.

3. The sexually transmitted disease shielding system of claim 1, wherein material is latex.

4. The sexually transmitted disease shielding system of claim 1, wherein material is from an animal.

5. The sexually transmitted disease shielding system of claim 4, wherein material is lambskin.

6. The sexually transmitted disease shielding system of claim 1, further comprising a receptacle at the tip end.

7. The sexually transmitted disease shielding system of claim 1, wherein the material is three times thicker at the base end of the wall than the material at the area of the tip end of the wall.

8. A method of reducing the risk of pregnancy and sexually transmitted diseases, the method comprising:
providing a sexually transmitted disease shielding system, the sexually transmitted disease shielding system comprising:
a condom having a body, the body having a wall made of a material, the wall having a base end, a tip end and a circumference at the base end, the material is thicker at the base end of the wall and the material is thinner at an area of the tip end of the wall, the wall is open at the base end for insertion of a penis and the wall is closed at a distal end; a pressure ring is formed around the circumference of the base end; and
a shield, the shield having a circular opening for accepting the penis and the shield having a retention ring formed around the circular opening;
before engaging in sex, a wearer:
pulling the shield over the shaft of the penis, the retention ring facing away from the wearer;
pulling the condom over a penis such that the pressure ring and base end cover a base portion of the penis and the tip end covers the tip portion of the penis; and
engaging the pressure ring into the retention ring.

9. The method of claim 8, wherein material is rubber.

10. The method of claim 8, wherein material is latex.

11. The method of claim 8, wherein material is from an animal.

12. The method of claim 11, wherein material is lambskin.

13. The method of claim 8, further comprising a receptacle at the tip end.

14. The method of claim 8, wherein the material is three times thicker at the base end of the wall than the material at the area of the tip end of the wall.

15. A sexually transmitted disease shielding system, the sexually transmitted disease shielding system comprising:
a condom having a body, the body having a wall made of a material, the wall having a base end, a tip end and a circumference at the base end, the material having a first thickness at the base end of the wall and the material having a second thickness at an area of the tip end of the wall whereas the first thickness is three times the second thickness, the wall is open at the base end for insertion of a penis and the wall is closed at a distal end; a pressure ring at the base end of the wall is formed around the circumference of the base end; and
a shield, the shield having a circular opening for accepting the penis and the shield has a retention ring formed around the circular opening;
whereas, the pressure ring of the condom fits in the retention ring of the shield and the pressure ring of the condom is removably held within the retention ring of the shield for the prevention of pregnancy and sexually transmitted disease.

16. The condom of claim 15, wherein material is rubber.

17. The condom of claim 15, wherein material is latex.

18. The condom of claim 15, wherein material is from an animal.

19. The condom of claim 18, wherein material is lambskin.

20. The condom of claim 15, further comprising a receptacle at the tip end.
